(19) United States
(12) Patent Application Publication
(10) Pub. No.: US 2023/0316156 A1
(43) Pub. Date: Oct. 5, 2023

(54) METHOD AND APPARATUS FOR LEARNING MULTI-LABEL ENSEMBLE BASED ON MULTI-CENTER PREDICTION ACCURACY

(71) Applicant: ELECTRONICS AND TELECOMMUNICATIONS RESEARCH INSTITUTE, Daejeon (KR)

(72) Inventors: Do Hyeun KIM, Daejeon (KR); Myung Eun LIM, Daejeon (KR); Jae Hun CHOI, Daejeon (KR)

(21) Appl. No.: 18/057,080

(22) Filed: Nov. 18, 2022

(30) Foreign Application Priority Data
Mar. 31, 2022 (KR) 10-2022-0040474

(51) Int. Cl. G06N 20/20

(52) U.S. Cl. CPC G06N 20/20

(57) ABSTRACT

Disclosed herein a method and apparatus for learning a multi-label ensemble based on multi-center prediction accuracy. According to an embodiment of the present disclosure, there is provided a multi-label ensemble learning method comprising: collecting a prediction value for learning data for each of a plurality of prediction models; calculating a prediction error of each of the prediction models using the prediction value of each of the prediction models and a correct answer prediction value; generating a weight label for each of the prediction models based on the prediction error; and learning an ensemble weight prediction model for predicting a weight of each of the prediction models using the weight label.

### CROSS REFERENCE TO RELATED APPLICATION

The present application claims priority to Korean Patent Application No. 10-2022-0040474, filed Mar. 31, 2022, the entire contents of which is incorporated herein for all purposes by this reference.

### BACKGROUND OF THE INVENTION

### 1. Field of the Invention

The present disclosure relates to a method and apparatus for learning a multi-label ensemble, and more particularly, to a method and apparatus for learning a multi-label ensemble based on multi-center prediction accuracy.

### 2. Description of the Related Art

The purpose of predicting a future health state by analyzing a patient's past health state history is to provide information to medical actors to help decision-making. To achieve this purpose, accurate prediction of the patient's future health state is essential, and several methods and techniques have been introduced for this purpose.

A method of increasing accuracy is to secure, systematize, and refine a large number of patients' health state histories. Due to the characteristics of artificial intelligence, the more data there is, the more sophisticated and huge models can be learned, and the more sophisticated prediction may be made by generalizing the data.

Another method is an approach that advances an artificial intelligence model structure to increase accuracy even in a situation in which limited data is given. A general method for upgrading and optimizing models for the purpose of improving accuracy is an ensemble technique. The ensemble technique is a technique for generating a new prediction result by synthesizing the prediction results of several predictors. Artificial intelligence technology using deep learning has high variability in prediction compared to other machine learning technologies. Therefore, the principle of increasing performance by introducing ensemble technology uses the point that performance increases by removing noises of prediction with high variability through arithmetic mean, weighted sum, or other synthesizing techniques of multiple prediction values.

### SUMMARY OF THE INVENTION

An object of the present disclosure is to provide a method and apparatus for learning a multi-label ensemble based on multi-center prediction accuracy.

Other objects and advantages of the present invention will become apparent from the description below and will be clearly understood through embodiments. In addition, it will be easily understood that the objects and advantages of the present disclosure may be realized by means of the appended claims and a combination thereof.

Disclosed herein a method and apparatus for learning a multi-label ensemble based on multi-center prediction accuracy. According to an embodiment of the present disclosure, there is provided a multi-label ensemble learning method comprising: collecting a prediction value for learning data for each of a plurality of prediction models; calculating a prediction error of each of the prediction models using the prediction value of each of the prediction models and a correct answer prediction value; generating a weight label for each of the prediction models based on the prediction error; and learning an ensemble weight prediction model for predicting a weight of each of the prediction models using the weight label.

According to the embodiment of the present disclosure, wherein the learning the ensemble weight prediction model comprises learning the ensemble weight prediction model so that the weight of each of the prediction models and the weight label are minimized.

According to the embodiment of the present disclosure, wherein the generating the weight label comprises: calculating error-based weight scores for the prediction models based on the prediction error; and generating the weight label based on the error-based weight scores.

According to the embodiment of the present disclosure, wherein the calculating the error-based weight scores comprises reflecting a first parameter value for adjusting a deviation between the error-based weight scores and calculating the error based weight scores for the prediction models based on the prediction error.

According to the embodiment of the present disclosure, wherein the generating the weight label based on the error-based weight scores comprises: optionally selecting at least some of error-based weight scores for the prediction models; and generating the weight label of each of the prediction models based on the at least some optionally selected error-based weight scores.

According to the embodiment of the present disclosure, wherein the generating the weight label of each of the prediction models comprises generating the weight label of each of the prediction models, by setting a sum of the at least some optionally selected error-based weight scores to 1 and setting the remaining error-based weight scores to 0 through a normalization process for the at least some optionally selected error-based weight scores.

According to the embodiment of the present disclosure, wherein the optionally selecting the at least some error-based weight scores comprises optionally selecting at least some of the error-based weight scores for the prediction models using a predetermined second parameter value.

According to the embodiment of the present disclosure, wherein the optionally selecting the at least some error-based weight scores comprises determining the number of prediction models using the second parameter value and the error-based weight scores for the prediction models and selecting an error-based weight score of a high value corresponding to the determined number of prediction models as the at least some error-based weight scores.

According to the embodiment of the present disclosure, wherein the generating the weight label of each of the prediction models comprises calculating a normalization threshold using the second parameter value and the at least some optionally selected error-based weight scores and generating the weight label of each of the prediction models using the normalization threshold, the second parameter value and the error-based weight scores for the prediction models.

According to another embodiment of the present disclosure, there is provided a multi-label ensemble learning method comprising: collecting a prediction value for learning data of each of prediction models; calculating a prediction error of each of the prediction models by comparing the prediction value of each of the prediction models and a correct answer prediction value; calculating error-based weight scores for the prediction models based on the prediction error; optionally selecting at least some of the error-based weight scores for the prediction models using a predetermined parameter value; and learning an ensemble weight prediction model for predicting a weight of each of the prediction models based on the at least some optionally selected error-based weight scores.

According to another embodiment of the present disclosure, there is provided a multi-label ensemble learning apparatus comprising: a collection unit configured to collect a prediction value for learning data for each of a plurality of prediction models; a generation unit configured to calculate a prediction error of each of the prediction models using the prediction value of each of the prediction models and a correct answer prediction value and to generate a weight label for each of the prediction models based on the prediction error; and a learning unit configured to learn an ensemble weight prediction model for predicting a weight of each of the prediction models using the weight label.

The features briefly summarized above with respect to the present disclosure are merely exemplary aspects of the detailed description below of the present disclosure, and do not limit the scope of the present disclosure.

According to the present disclosure, it is possible to provide a method and apparatus for learning a multi-label ensemble based on multi-center prediction accuracy.

Effects obtained in the present disclosure are not limited to the above-mentioned effects, and other effects not mentioned above may be clearly understood by those skilled in the art from the following description.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a flowchart of a multi-label ensemble learning method according to an embodiment of the present disclosure;

FIG. 2 is a detailed flowchart of step S140 of FIG. 1;

FIG. 3 is a diagram for explaining a process of generating a weight label;

FIG. 4 is a diagram for explaining a method of calculating an error between a prediction and a future state;

FIG. 5 is a diagram for explaining a method of calculating an error-based weight score;

FIG. 6 is a diagram for explaining a gradient change according to the magnitude of an exponential function constant;

FIG. 7 is a diagram for explaining a weight score selection and normalization process.

FIG. 8 is a diagram for explaining a method of generating a weight label obtained by modifying a sparse-max function;

FIG. 9 is a diagram illustrating a configuration of a multi-label ensemble learning apparatus according to an embodiment of the present disclosure; and

FIG. 10 is a diagram illustrating a configuration of a device to which the multi-label ensemble learning apparatus according to an embodiment of the present disclosure is applied.

### DESCRIPTION OF THE PREFERRED EMBODIMENTS

Hereinafter, exemplary embodiments of the present disclosure will be described in detail with reference to the accompanying drawings so that those skilled in the art may easily implement the present disclosure. However, the present disclosure may be implemented in various different ways, and is not limited to the embodiments described therein.

In describing exemplary embodiments of the present disclosure, well-known functions or constructions will not be described in detail since they may unnecessarily obscure the understanding of the present disclosure. The same constituent elements in the drawings are denoted by the same reference numerals, and a repeated description of the same elements will be omitted.

In the present disclosure, when an element is simply referred to as being “connected to”, “coupled to” or “linked to” another element, this may mean that an element is “directly connected to”, “directly coupled to” or “directly linked to” another element or is connected to, coupled to or linked to another element with the other element intervening therebetween. In addition, when an element “includes” or “has” another element, this means that one element may further include another element without excluding another component unless specifically stated otherwise.

In the present disclosure, elements that are distinguished from each other are for clearly describing each feature, and do not necessarily mean that the elements are separated. That is, a plurality of elements may be integrated in one hardware or software unit, or one element may be distributed and formed in a plurality of hardware or software units. Therefore, even if not mentioned otherwise, such integrated or distributed embodiments are included in the scope of the present disclosure.

In the present disclosure, elements described in various embodiments do not necessarily mean essential elements, and some of them may be optional elements. Therefore, an embodiment composed of a subset of elements described in an embodiment is also included in the scope of the present disclosure. In addition, embodiments including other elements in addition to the elements described in the various embodiments are also included in the scope of the present disclosure.

In the present document, such phrases as ‘A or B’, ‘at least one of A and B’, ‘at least one of A or B’, ‘A, B or C’, ‘at least one of A, B and C’ and ‘at least one of A, B or C’ may respectively include any one of items listed together in a corresponding phrase among those phrases or any possible combination thereof.

Three approaches of the existing ensemble technique representatively include selective ensemble, weighted sum ensemble, and stacking ensemble. The selective ensemble is technology in which, when data is divided into multiple domains, a model having the highest accuracy is selected from among multiple predictor models in a corresponding domain through a validation set, and then a test set is predicted using the model selected in the domain corresponding to the test set. The selective ensemble can improve accuracy by selecting an effective model according to the domain when there is a clear correlation between the domain and model accuracy. However, if the correlation is not high, since a model with low performance may be selected, accuracy may be lowered.

The weighted sum ensemble is technology of calculating N weights a1, a2, a3, . . . , aN with respect to prediction values p1, p2, p3, . . . , pN from N individual predictors and calculating a final ensemble prediction value pE using their weighted sum, that is, p_{E}=Σᵢ₌₁^{N}pᵢ*aᵢ. A method of obtaining a weight includes a learning method using deep learning and a method of obtaining a weight using an algorithm. This has an advantage in that accuracy increases by synthesizing multiple predictions to reduce variability, but has a disadvantage in that predictions with large errors are summed to contribute to occurrence of errors.

In the stacking ensemble, prediction values p1, p2, p3, . . . , pN from individual predictors are input to a deep learning ensemble apparatus to immediately generate a new ensemble prediction value pE. In this case, since an ensemble apparatus immediately generates an ensemble prediction value, it is difficult to determine on what basis a prediction value is generated or which prediction value is selected according to the situation.

Artificial intelligence learning through supervised learning requires input data and a correct answer label. In artificial intelligence, input data is received, a prediction value is compared with the correct answer label and optimization is performed in a direction of minimizing errors. Therefore, the clearer the correct answer label, the more efficient artificial intelligence learning can be performed. However, there are two ambiguities in an ensemble weight learning label through multi-center prediction.

First, it is ambiguous as to which prediction value is set as the correct answer to participate in the ensemble and how many prediction values are considered as the correct answer depending on the situation. In the case of the selective ensemble, when the number of predictors is N, like [L1, L2, L3, L4, L5] (Ln=whether to select n^{th} prediction)=[1, 0, 0, 0, 1], by assigning 1 to a label corresponding to a predictor that shall be selected and assigning 0 to a predictor that shall not be selected, artificial intelligence is trained to predict as closely as possible to this label. However, depending on the situation, if a number of prediction values are close to the actual patient's future state, it is quite ambiguous as to which prediction value is set as correct answers and how many prediction values are set as correct answers. In particular, if it is learned to select one prediction value that is close to a patient's future state as the correct answer in a fixed manner, since the correct answer may be changed frequently depending on the noise of the data, it is highly likely that noise is learned, rather than that the characteristics of a predictor which provides the closest prediction value are learned. In addition, if predictors close to k correct answers are selected in a fixed manner and only one predictor provides an accurate prediction, k−1 predictors act as noise to the ensemble predictor. Therefore, it is necessary to flexibly adjust the number of correct answer predictors and selection of the correct answer predictors according to the situation.

Second, there is ambiguity about how large weight of the correct answer label is given. For example, in the case of predictors 1 and 5 selected as correct answers [L1, L2, L3, L4, L5] (Ln=whether to select n^{th} prediction)=[1, 0, 0, 0, 1], if predictor 1 is 30% more accurate than predictor 5, the artificial intelligence learns that both 1 and 5 are equal because they have the same correct answer. Therefore, since a larger weight is not given to predictor 1, an error may occur. Also, when an error of 30% occurs, it is ambiguous as to which numerical value will be reflected in the correct answer ensemble weight as in [1, 0, 0, 0, 0.7].

The embodiments of the present disclosure are to resolve ambiguity of an ensemble weight correct answer label by obtaining an error between the prediction of each predictor and the prediction of an actual future patient state, determining whether the prediction is included in the ensemble correct answer or not depending on the degree of error, and putting differentiation even in the same correct answer by having consecutive values according to the error rather than dichotomous division of 0 and 1.

In this case, in the embodiments of the present disclosure, an ensemble weight prediction model that outputs a weight of each prediction model may be learned, by giving a high weight to accurate prediction and giving a low weight to inaccurate prediction among prediction values generated by multiple future state predictors (or prediction models). The weights given to the predictors or the predictive models may be utilized when generating an ensemble predictor.

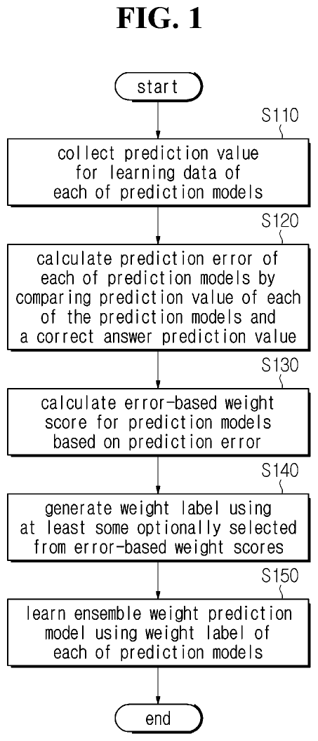

FIG. 1 is a flowchart of a multi-label ensemble learning method according to an embodiment of the present disclosure.

Referring to FIG. 1, the multi-label ensemble learning method according to the embodiment of the present disclosure includes step S110 of collecting a prediction value for learning data of each of prediction models (or predictors), step S120 of calculating a prediction error of each of the prediction models by comparing a prediction value of each of the prediction models and a correct answer prediction value, step S130 of calculating error-based weight scores for the prediction models based on the prediction error, step S140 of optionally selecting at least some of error-based weight scores for the prediction models using a predetermined parameter value and generating a weight label using the at least some optionally selected error-based weight scores, and step S150 of learning an ensemble weight prediction model which predicts the weight of each of the prediction models using the weight label of each of the prediction models.

Step S110 is a process of collecting the prediction values using learning data from each of multi-center predictors for giving weights. A future state prediction value is generated from a future state predictor, and past state time series data [x1, x2, x3, . . . , xt] up to an arbitrary time t is received, and a future state prediction value P is generated. The same past state time-series data [x1, x2, x3, . . . , xt] is input for N future state predictors to collect N future state prediction values [p1, p2, p3, . . . , pN]t+1. [p1, p2, p3, . . . , pN]t+1 consists of N prediction values [p1, p2, p3, . . . , pN], because there are N predictors, and the prediction value is denoted by [p1, p2, p3, . . . , pN]t+1 because it is future state prediction for a time t+1.

That is, step S110 is a process of, at each predictor, predicting the prediction value at the time t+1 from learning data by using the time series data up to the time t and collecting the prediction values predicted in this way. Depending on the situation, in step S110, in addition to 1, . . . , t data, using partial time series data such as 1, 2 data or 1, 2, 3 data, time series prediction values at 2, . . . , t+1 times may be collected.

Here, the predictor may mean a machine learning prediction model trained with its own data, and the prediction model may be composed of LSTM, which is one of the deep neural network structures, and receives time series data such as blood pressure, cholesterol, and blood sugar as input and calculate or return a future state prediction value.

Step S120 is a process of calculating an error between a future state correct answer prediction value yt+1 and [p1, p2, p3, . . . , pN]t+1 at an actual time t+1 and may include a method of calculating an absolute error such as |y−pn| and a method of using the square of an error such as |y−pn|2. In this case, in step S120, errors [e1, e2, e3, . . . , eN]t+1 equal in number to the number of future generation predictors may be generated.

Steps S130 and S140 are a process of generating a weight label based on the magnitude of the prediction error. The larger the prediction error, the lower the weight is given to the prediction value, and the smaller the prediction error, the higher the weight is given to the prediction value. At this time, in step S140, similarly to the process of generating the prediction error in step S120, weight labels [l1, l2, l3, . . . , lN] t+1 equal in number to the number of future generation predictors may be generated.

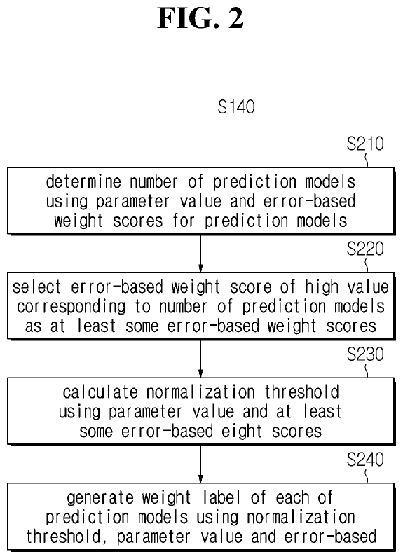

In some embodiments, in step S140, as shown in FIG. 2, the number of prediction models is determined using a predetermined parameter value and error-based weight scores for the prediction models, and an error-based weight score of a high value corresponding to the determined number of prediction models is selected as at least some error-based weight scores (S210 and S220).

Here, the parameter value is a value for optionally selecting the error-based weight score, and the number of error-based weight scores that are optionally selected through adjustment of the corresponding value varies. Therefore, the number of weight labels applied to learn the ensemble weight prediction model varies. That is, embodiments of the present disclosure may optionally select the number of prediction models with high prediction accuracy used when learning the ensemble weight prediction model through the parameter value, thereby increasing the prediction accuracy of the ensemble weight prediction model.

When at least some error-based weight scores are optionally selected in step S220, a normalization threshold is calculated using the parameter value and the at least some optionally selected error-based weight scores, and the weight label of each of the prediction models is generated using the normalization threshold, the parameter value, and the error-based weight scores for the prediction models (S230 and S240).

Step S150 is a process of learning an ensemble weight prediction model M that predicts a weight for the prediction value of each of the prediction models according to the weight label generated in step S140. The ensemble weight prediction model M receives a future state prediction value [p1, p2, p3, . . . , pN]t+1 at a time t+1 or k+1 future state prediction values [p1, p2, p3, . . . , pN]t+1−k˜[p1, p2, p3, . . . , pN]t+1 up to a time t+1 as input and predicts a weight [a1, a2, a3, . . . , aN] t+1 for [p1, p2, p3, . . . , pN]t+1. Input for the ensemble weight prediction model M may include past state time series data [x1, x2, x3, . . . , xt] up to a time t. The ensemble weight prediction model M may be learned such that a difference between a weight [a1, a2, a3, . . . , aN] t+1 for the prediction value [p1, p2, p3, . . . , pN]t+1 of each of the prediction models and a weight label [l1, l2, l3, . . . , lN] t+1 is minimized.

Here, the weight [a1, a2, a3, . . . , aN] t+1 for the prediction value [p1, p2, p3, . . . , pN]t+1 of each of the prediction models is a score meaning accuracy or importance of the prediction model for each prediction result (prediction value) and means that the higher the weight, the closer the prediction value of the prediction model is to the correct answer.

The ensemble weight prediction model may be configured to receive the prediction value of each prediction model and a time series record (or learning data) as input and to output a weight. For example, the ensemble weight prediction model may be composed of a deep neural network (DNN) model that outputs a prediction value p of a prediction model at a time t+1 and a weight for learning data at a time t or an RNN or LSTM model that outputs partial time-series prediction values at 2, . . . , t+1 and a weight for time series input. The weight may be calculated as a ratio of each prediction model prediction value to a sum of errors between the prediction model prediction value and a measured value (or correct answer prediction value).

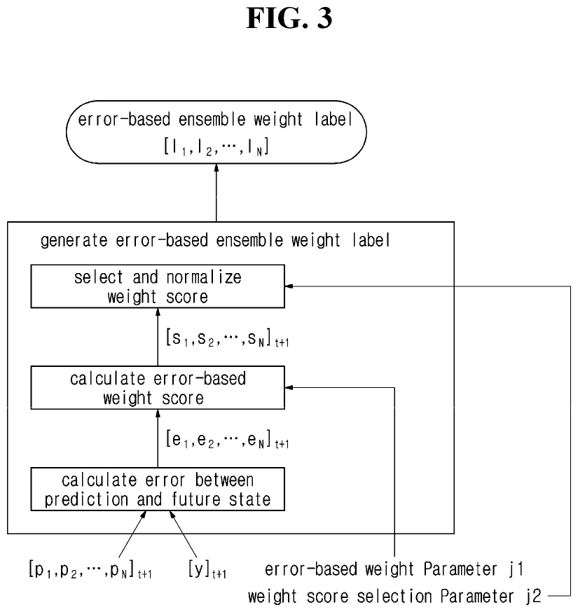

A multi-label ensemble learning method according to an embodiment of the present disclosure, which is learned through this process, will be described in detail with reference to FIGS. 3 to 8.

The multi-label ensemble learning method according to the embodiment of the present disclosure includes a process of collecting future patient state prediction values from each of the predictors using ensemble learning data, a process of generating an error-based ensemble weight label, and a process of learning an ensemble weight prediction model using the ensemble weight label.

The ensemble learning data includes a patient state [x1, x2, x3, . . . , xt] before a prediction time and a patient state yt+1 at the prediction time. Predictors (or prediction models) that predict future patient state prediction values receive the patient state [x1, x2, x3, . . . , xt] before the prediction time as input from the ensemble learning data, and predict the future patient state prediction value [p1, p2, p3, . . . , pN]t+1 at the prediction time. In the error-based ensemble weight label generation process, the future state prediction value [p1, p2, p3, . . . , pN]t+1 and the patient state yt+1 at the prediction time are compared to generate an ensemble weight label [l1, l2, l3, . . . , lN] t+1. In the process of learning the ensemble weight prediction model, the patient state [x1, x2, x3, . . . , xt] before the prediction time and the future state prediction value [p1, p2, p3, . . . , pN]t+1 or k+1 future state prediction values [p1, p2, p3, . . . , pN]t+1−k to [p1, p2, p3, . . . , pN]t+1 up to the time t+1 are received as input to enable the ensemble weight prediction model to learn the weight [a1, a2, a3, . . . , aN]t+1 for the future state prediction value. In this case, the ensemble weight predicted by the ensemble weight prediction model is learned in a direction in which an error with the error-based ensemble weight label is optimized.

FIG. 3 is a diagram for explaining a process of generating a weight label, which shows a process of generating an error-based ensemble weight label.

As shown in FIG. 3, the process of generating the error-based ensemble weight label includes a process of calculating an error between a prediction and a future state, a process of calculating an error-based weight score, and a process of generating an error-based ensemble weight label by selecting and normalizing the weight score.

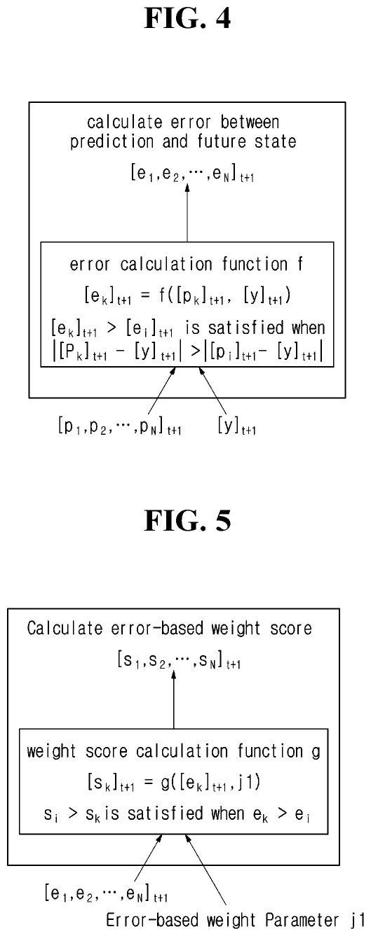

In the process of calculating the error between the prediction and the future state, as shown in FIG. 4, a prediction error [e1, e2, e3, . . . , eN]t+1 between a future state prediction value [p1, p2, p3, . . . , pN]t+1 and the patient state yt+1 at the prediction time is calculated. In this case, the method of calculating the error may include a method of calculating an absolute error such as |y−pn|, a method of using the square of the error such as |y−pn|2, etc. The larger the difference between the prediction value and the patient state, the larger the prediction error e may be calculated. That is, for a given function f(x), when |y−pa|>|y−pb|, if the property of f(|y−pa|)>f(|y−pb|) is satisfied, f(x) may be used as the error calculation function. At this time, the prediction error [e1, [e1, e2, e3, . . . , eN]t+1 equal in number to the number of future generation predictors may be generated as a result of the error calculation function f(x).

In the process of calculating the error-based weight score, as shown in FIG. 5, the error-based weight score is calculated by receiving the generated prediction error as input. It means that, as the error-based weight score for the prediction value increases, the prediction is accurate and the error is small and, as the error-based weight score for the prediction value decreases, the prediction is inaccurate and the error is large. Accordingly, the smaller the prediction error, the higher the error-based weight score and the larger the prediction error, the lower the error-based weight score. That is, there is an inverse relationship between the prediction error and the error-based weight score. In this case, in the error-based weight score, a deviation between the error-based weight scores may be adjusted by an error-based weight parameter value j1. In other words, in the embodiments of the present disclosure, the error-based weight scores for the prediction models may be calculated by reflecting the parameter value j1 for adjusting the deviation between the error-based weight scores. The error-based weight score [s1, s2, s3, . . . , sN] t+1 may be calculated by [Equation 1] below.

$${[ {s}_{1} , {s}_{2} , {s}_{3} … {s}_{N} ]}_{t + 1} = [ \frac{1}{{e}^{{e}_{1} * j 1}} , \frac{1}{{e}^{{e}_{2} * j 1}} , … \frac{1}{{e}^{{e}_{N} * j 1}} ] \qquad [ Equation 1 ]$$

where, e may denote a natural constant, and eN may denote a prediction error of an N^{th} predictor.

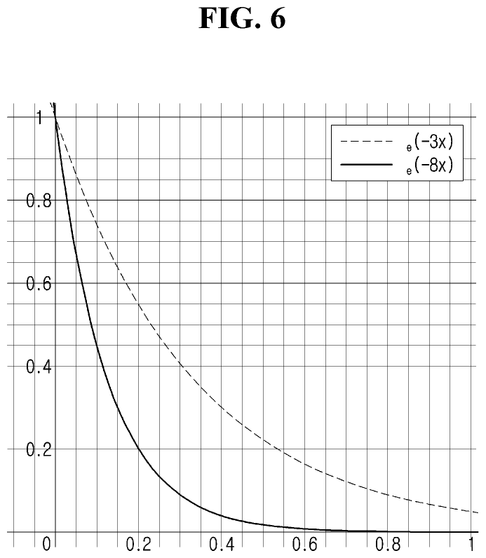

As shown in FIG. 6, due to the characteristics of the exponential function, as the parameter value j1 increases, the amount of change in y according to x increases. Therefore, the greater the difference between prediction errors, the greater the score difference. That is, if it is desired to learn to evenly give a weight to a large number of prediction values, the parameter value j1 may be decreased, and, when it is desired to learn to focus the weight on a small number of prediction values with small prediction errors, the parameter value j1 may be increased.

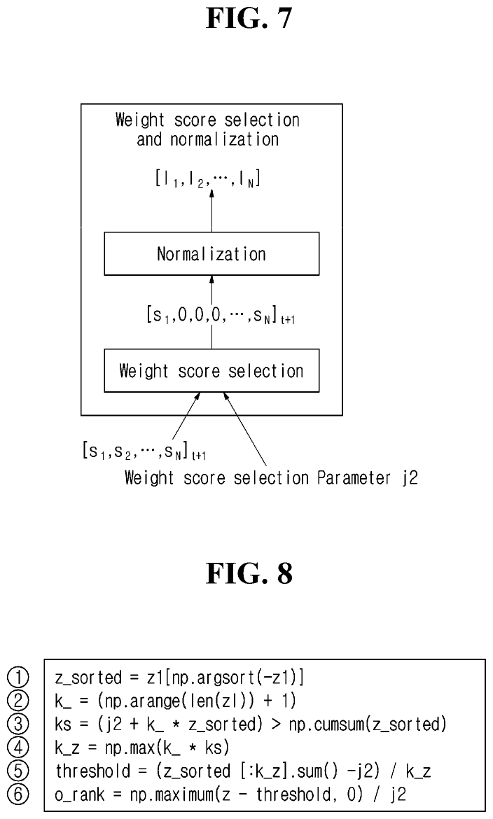

In the process of selecting and normalizing the error-based weight score, as shown in FIG. 7, after the error-based weight score is optionally selected from the generated error-based weight score [s1, s2, s3, . . . , sN]t+1, the sum of all the error-based weight scores is set to 1 to generate an ensemble weight label [l1, l2, l3, . . . , lN]t+1. The reason for optionally selecting the weight score is to generate a weight label in which a score is given to a prediction value considered to be accurate, because a partial score is given to even inaccurate prediction. Selection is performed by giving a weight label of 0 to a prediction value considered to be inaccurate and normalization is performed so that a sum of weight labels for all prediction values is 1.

That is, in the process of selecting and normalizing the error-based weight score, the sum of at least some error-based weight scores optionally selected through a normalization process for the at least some optionally selected error-based weight scores is set to 1 and the remaining error-based weight scores str set to 0, thereby generating an ensemble weight label for each of the prediction models.

In this case, the ensemble weight label may be generated based on sparse-max to which the parameter value j2 is applied. The sparse-max algorithm is an algorithm that re-evaluates importance based on a difference between weights for weights, performs recalculation so that a relatively small weight is replaced with 0 and a sum of large weights becomes 1. In the embodiments of the present disclosure, by applying the parameter value j2 to the sparse-max algorithm, the weight label by the parameter value j2 may be calculated. Specifically, in the embodiments of the present disclosure, by variabalizing the parameter value j2 to modify the sparse-max algorithm, the weight label may be generated according to the parameter value j2. In the sparse-max basic algorithm, the value on the parameter is fixed, but when the input weighted sum is 1, the optimal number of combinations is maximized. In an actual situation, this value needs to be variably changed according to the scale of the weight and data. In the embodiment of the present disclosure, as the parameter value j2 increases, a plurality of models participate in generating the weight label and, as the parameter value j2 decreases, a smaller number of models participate in generating the weight label. In addition, even in the normalization process, the smaller the parameter value j2, the larger the deviation between weight labels and the larger the parameter value j2, the smaller the deviation between the weight labels. In addition, the parameter value j2 may be directly determined in consideration of the above-described characteristics when designing the ensemble model. That is, in the embodiments of the present disclosure, the criterion for the number of error-based weight scores to be replaced with 0 may be adjusted through the parameter value j2.

In some embodiments, in the process of selecting and normalizing the error-based weight score, as shown in FIG. 8, the error-based weight score (z1=[s1, s2, s3, . . . , sN] t+1) is sorted (z_sorted) in descending order.

A value k_z that determines how many error-based weight scores are kept as non-zero values is calculated. For example, if k_z=3, all the error-based weight scores except for 3 are replaced with 0. In a method of obtaining the value k_z, after the error-based weight scores sorted in descending order are multiplied by the rank in the magnitude of the sorted error-based weight scores by matrix element, when the parameter value j2 is added to each element, it is set to a number greater than the cumulative sum of the sorted error-based weight scores. That is, as shown in the third and fourth steps of FIG. 8, the number k_z of prediction models may be determined using the error-based weight scores for the prediction models sorted in descending order and the parameter value, and the error-based weight score having an upper-rank value corresponding to the determined number of prediction models may be optionally selected.

Thereafter, a normalization threshold is obtained. As shown in the fifth step of FIG. 8, the normalization threshold may be calculated by subtracting the parameter value j2 from the sum of the k_z upper-rank error-based weight scores in the order of magnitude and then dividing it by the value k_z.

As shown in the sixth step of FIG. 8, a final normalized ensemble weight label [l1, l2, l3, . . . , lN]t+1 may be generated by subtracting the normalization threshold from the error-based weight score z and then dividing it by the parameter value j2. In this case, the ensemble weight label [l1, l2, l3, . . . , lN]t+1 may be normalized so that only k_z upper-rank error-based weight scores are not 0 in the order of magnitude and the sum of the remaining error-based weight labels is 1. That is, the final normalized ensemble weight label [l1, l2, l3, . . . , lN]t+1 is normalized so that the sum of the weight labels corresponding in number to the value k_z is 1 and the remaining weight labels have a value of 0, thereby learning the ensemble weight prediction model using the weight label having a non-zero value. Accordingly, in the embodiments of the present disclosure, by excluding the prediction value of the prediction mode with low accuracy and optionally selecting only the prediction value of the prediction model with high accuracy to generate the weight label, the ensemble weight prediction model may be learned using the generated weight label. Therefore, by outputting an ensemble weight with high accuracy, it is possible to provide a more accurate ensemble prediction result.

As such, in the multi-label ensemble learning method according to the embodiments of the present disclosure, by giving a high weight to an accurate prediction and giving a low weight to an inaccurate prediction among prediction values generated by several future state predictors (or prediction models), the ensemble weight prediction model that outputs the weight of each prediction model may be learned.

In addition, in the multi-label ensemble learning method according to the embodiments of the present disclosure, by synthesizing the prediction results of future health state predictors of a plurality of medical institutions learned independently to perform more sophisticated prediction, it is possible to support clinical decision-making of a medical personnel.

In addition, in the multi-label ensemble learning method according to the embodiments of the present disclosure, in future health ensemble prediction for clinical decision-making support, it is possible to improve accuracy by selectively learning to give a weight to only accurate predictions through error-based labeling to prevent inaccurate predictions from participating in the ensemble.

In addition, in the multi-label ensemble learning method according to the embodiments of the present disclosure, it is possible to learn whether to reduce variability by allowing a plurality of predictions to participate in the ensemble or allowing only a small number of accurate predictions to participate in the ensemble through adjustment of the parameter value, thereby providing a flexible ensemble according to the situation.

In addition, in the multi-label ensemble learning method according to the embodiments of the present disclosure, in giving an existing weight, since the criterion for which prediction is given a high weight is unclear, it is difficult to interpret a learning result and reliability is lowered. However, by giving a weight based on a clear criterion such as error, it is possible to resolve ambiguity of the criterion for the weight and to provide interpretation of the given weight.

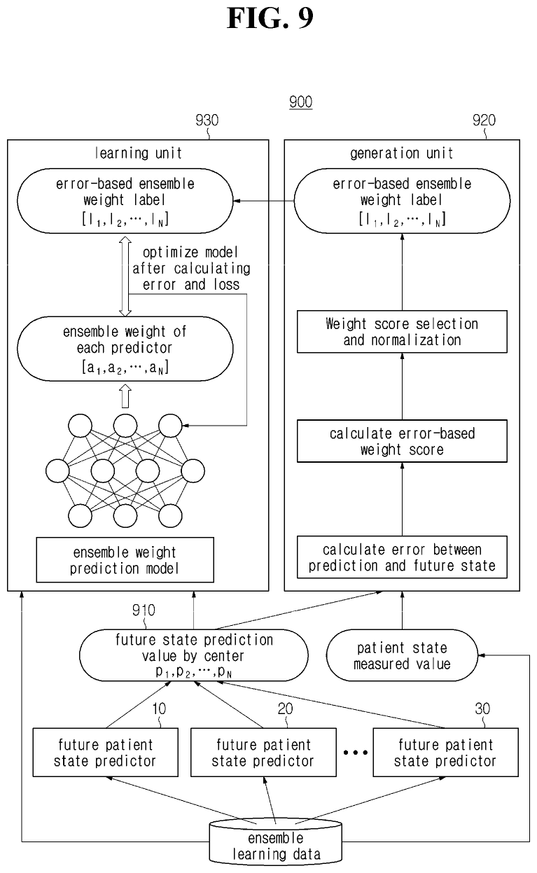

FIG. 9 is a diagram illustrating a configuration of a multi-label ensemble learning apparatus according to an embodiment of the present disclosure, which shows a configuration of the apparatus for performing the method of FIGS. 1 to 8.

Referring to FIG. 9, the multi-label ensemble learning apparatus 900 according to the embodiment of the present disclosure includes a collection unit 910, a generation unit 920 and a learning unit 930.

The collection unit 910 collects a prediction value [p1, p2, p3, . . . , pN]t+1 predicted by each of the future patient state predictors (prediction models) 10, 20 and 30 using time series learning data stored in ensemble learning data.

The generation unit 920 calculates a prediction error of each of the prediction models using a prediction value of each of the prediction models and a correct answer prediction value (measured value of a patient state), calculates the error-based weight scores for the prediction models based on the prediction error, and generate a weight label for each of the prediction models based on the error-based weight scores.

In this case, the generation unit 920 may optionally select at least some of the error-based weight scores for the prediction models 10, 20 and 30 and generate the weight label of each of the prediction models based on the at least some optionally selected error-based weight scores.

In this case, the generation unit 920 may generate the weight label of each of the prediction models, by setting the sum of the at least some optionally selected error-based weight scores to 1 and setting the remaining error-based weight scores to 0 through the normalization process for the at least some optionally selected error-based weight scores.

In this case, the generation unit 920 may optionally select at least some of the error-based weight scores for the prediction models 10, 20 and 30 using a predetermined parameter value j2, determining the number of prediction models using the parameter value j2 and the error-based weight scores for the prediction models, and selecting the error-based weight score of a high value corresponding to the determined number of prediction models as the at least some error-based weight scores.

In some embodiments, the generation unit 920 may calculate a normalization threshold using the parameter value j2 and the at least some optionally selected error-based weight scores, and generate the weight label for each of the prediction models 10, and 30 using the normalization threshold, the parameter value j2 and the error-based weight scores for the prediction models.

The learning unit 930 receives the error-based weight label of each of the prediction models 10, 20 and 30 generated by the generation unit 920 and the ensemble learning data, and learn the ensemble weight prediction model so that an error of an ensemble weight of each predictor output from the ensemble weight prediction model is minimized.

Although the description is omitted from FIG. 9, the multi-label ensemble learning apparatus according to the embodiment of the present disclosure may include all the contents described with reference to FIGS. 1 to 8, which will be apparent to those skilled in the art.

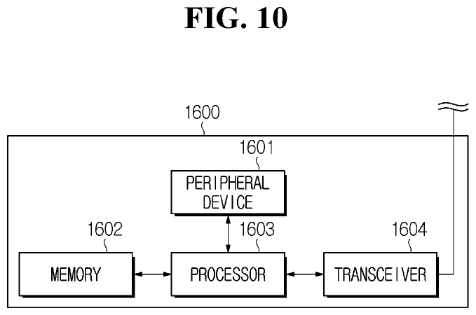

FIG. 10 is a diagram illustrating a configuration of a device to which the multi-label ensemble learning apparatus according to an embodiment of the present disclosure is applied.

An embodiment of the multi-label ensemble learning apparatus 900 of FIG. 9 may be a device 1600 of FIG. 10. Referring to FIG. 10, the device 1600 may include a memory 1602, a processor 1603, a transceiver 1604 and a peripheral device 1601. In addition, for example, the device 1600 may further include another configuration and is not limited to the above-described embodiment.

More specifically, the device 1600 of FIG. 10 may be an exemplary hardware/software architecture such as an ensemble learning system, an ensemble prediction device and a decision support device. Herein, as an example, the memory 1602 may be a non-removable memory or a removable memory. In addition, as an example, the peripheral device 1601 may include a display, GPS or other peripherals and is not limited to the above-described embodiment.

In addition, as an example, like the transceiver 1604, the above-described device 1600 may include a communication circuit. Based on this, the device 1600 may perform communication with an external device.

In addition, as an example, the processor 1603 may be at least one of a general-purpose processor, a digital signal processor (DSP), a DSP core, a controller, a micro controller, application specific integrated circuits (ASICs), field programmable gate array (FPGA) circuits, any other type of integrated circuit (IC), and one or more microprocessors related to a state machine. In other words, it may be a hardware/software configuration playing a controlling role for controlling the above-described device 1600. In addition, the processor 1603 may be performed by modularizing the functions of the generation unit 920 and the learning unit 930 of FIG. 9.

Herein, the processor 1603 may execute computer-executable commands stored in the memory 1602 in order to implement various necessary functions of the multi-label ensemble learning apparatus. As an example, the processor 1603 may control at least any one operation among signal coding, data processing, power controlling, input and output processing, and communication operation. In addition, the processor 1603 may control a physical layer, an MAC layer and an application layer. In addition, as an example, the processor 1603 may execute an authentication and security procedure in an access layer and/or an application layer but is not limited to the above-described embodiment.

In addition, as an example, the processor 1603 may perform communication with other devices via the transceiver 1604. As an example, the processor 1603 may execute computer-executable commands so that the multi-label ensemble learning apparatus may be controlled to perform communication with other devices via a network. That is, communication performed in the present invention may be controlled. As an example, the transceiver 1604 may send a RF signal through an antenna and may send a signal based on various communication networks.

In addition, as an example, MIMO technology and beam forming technology may be applied as antenna technology but are not limited to the above-described embodiment. In addition, a signal transmitted and received through the transceiver 1604 may be controlled by the processor 1603 by being modulated and demodulated, which is not limited to the above-described embodiment.

While the exemplary methods of the present disclosure described above are represented as a series of operations for clarity of description, it is not intended to limit the order in which the steps are performed, and the steps may be performed simultaneously or in different order as necessary. In order to implement the method according to the present disclosure, the described steps may further include other steps, may include remaining steps except for some of the steps, or may include other additional steps except for some of the steps.

The various embodiments of the present disclosure are not a list of all possible combinations and are intended to describe representative aspects of the present disclosure, and the matters described in the various embodiments may be applied independently or in combination of two or more.

In addition, various embodiments of the present disclosure may be implemented in hardware, firmware, software, or a combination thereof. In the case of implementing the present invention by hardware, the present disclosure can be implemented with application specific integrated circuits (ASICs), Digital signal processors (DSPs), digital signal processing devices (DSPDs), programmable logic devices (PLDs), field programmable gate arrays (FPGAs), general processors, controllers, microcontrollers, microprocessors, etc.

The scope of the disclosure includes software or machine-executable commands (e.g., an operating system, an application, firmware, a program, etc.) for enabling operations according to the methods of various embodiments to be executed on an apparatus or a computer, a non-transitory computer-readable medium having such software or commands stored thereon and executable on the apparatus or the computer.

What is claimed is:

1. A multi-label ensemble learning method comprising:
collecting a prediction value for learning data for each of a plurality of prediction models;
calculating a prediction error of each of the prediction models using the prediction value of each of the prediction models and a correct answer prediction value;
generating a weight label for each of the prediction models based on the prediction error; and
learning an ensemble weight prediction model for predicting a weight of each of the prediction models using the weight label.

2. The multi-label ensemble learning method of claim 1, wherein the learning the ensemble weight prediction model comprises learning the ensemble weight prediction model so that the weight of each of the prediction models and the weight label are minimized.

3. The multi-label ensemble learning method of claim 1, wherein the generating the weight label comprises:
calculating error-based weight scores for the prediction models based on the prediction error; and
generating the weight label based on the error-based weight scores.

4. The multi-label ensemble learning method of claim 3, wherein the calculating the error-based weight scores comprises reflecting a first parameter value for adjusting a deviation between the error-based weight scores and calculating the error based weight scores for the prediction models based on the prediction error.

5. The multi-label ensemble learning method of claim 3, wherein the generating the weight label based on the error-based weight scores comprises:
optionally selecting at least some of error-based weight scores for the prediction models; and
generating the weight label of each of the prediction models based on the at least some optionally selected error-based weight scores.

6. The multi-label ensemble learning method of claim 5, wherein the generating the weight label of each of the prediction models comprises generating the weight label of each of the prediction models, by setting a sum of the at least some optionally selected error-based weight scores to 1 and setting the remaining error-based weight scores to 0 through a normalization process for the at least some optionally selected error-based weight scores.

7. The multi-label ensemble learning method of claim 5, wherein the optionally selecting the at least some error-based weight scores comprises optionally selecting at least some of the error-based weight scores for the prediction models using a predetermined second parameter value.

8. The multi-label ensemble learning method of claim 7, wherein the optionally selecting the at least some error-based weight scores comprises determining the number of prediction models using the second parameter value and the error-based weight scores for the prediction models and selecting an error-based weight score of a high value corresponding to the determined number of prediction models as the at least some error-based weight scores.

9. The multi-label ensemble learning method of claim 8, wherein the generating the weight label of each of the prediction models comprises calculating a normalization threshold using the second parameter value and the at least some optionally selected error-based weight scores and generating the weight label of each of the prediction models using the normalization threshold, the second parameter value and the error-based weight scores for the prediction models.

10. A multi-label ensemble learning method comprising:
collecting a prediction value for learning data of each of prediction models;
calculating a prediction error of each of the prediction models by comparing the prediction value of each of the prediction models and a correct answer prediction value;
calculating error-based weight scores for the prediction models based on the prediction error;
optionally selecting at least some of the error-based weight scores for the prediction models using a predetermined parameter value; and
learning an ensemble weight prediction model for predicting a weight of each of the prediction models based on the at least some optionally selected error-based weight scores.

11. The multi-label ensemble learning method of claim 10, further comprising generating the weight label of each of the prediction models, by setting a sum of the at least some optionally selected error-based weight scores to 1 and setting the remaining error-based weight scores to 0 through a normalization process for the at least some optionally selected error-based weight scores,
wherein the learning the ensemble weight prediction model comprises learning the ensemble weight prediction model using the weight label of each of the prediction models.

12. The multi-label ensemble learning method of claim 10, wherein the optionally selecting the at least some error-based weight scores comprises determining the number of prediction models using the parameter value and the error-based weight scores for the prediction models and selecting an error-based weight score of a high value corresponding to the determined number of prediction models as the at least some error-based weight scores.

13. The multi-label ensemble learning method of claim 12, further comprising calculating a normalization threshold using the parameter value and the at least some optionally selected error-based weight scores and generating the weight label of each of the prediction models using the normalization threshold, the parameter value and the error-based weight scores for the prediction models,
wherein the learning the ensemble weight prediction mode comprises learning the ensemble weight prediction model using the weight label of each of the prediction models.

14. A multi-label ensemble learning apparatus comprising:
a collection unit configured to collect a prediction value for learning data for each of a plurality of prediction models;
a generation unit configured to calculate a prediction error of each of the prediction models using the prediction value of each of the prediction models and a correct answer prediction value and to generate a weight label for each of the prediction models based on the prediction error; and
a learning unit configured to learn an ensemble weight prediction model for predicting a weight of each of the prediction models using the weight label.

15. The multi-label ensemble learning apparatus of claim 14, wherein the generation unit is configured to:
calculate error-based weight scores for the prediction models based on the prediction error; and
generate the weight label based on the error-based weight scores.

16. The multi-label ensemble learning apparatus of claim 15, wherein the generation unit is configured to:
optionally select at least some of error-based weight scores for the prediction models; and
generate the weight label of each of the prediction models based on the at least some optionally selected error-based weight scores.

17. The multi-label ensemble learning apparatus of claim 16, wherein the generation unit is configured to generate the weight label of each of the prediction models, by setting a sum of the at least some optionally selected error-based weight scores to 1 and setting the remaining error-based weight scores to 0 through a normalization process for the at least some optionally selected error-based weight scores.

18. The multi-label ensemble learning apparatus of claim 16, wherein the generation unit is configured to optionally select at least some of the error-based weight scores for the prediction models using a predetermined parameter value.

19. The multi-label ensemble learning apparatus of claim 18, wherein the generation unit is configured to determine the number of prediction models using the parameter value and the error-based weight scores for the prediction models and to select an error-based weight score of a high value corresponding to the determined number of prediction models as the at least some error-based weight scores.

20. The multi-label ensemble learning apparatus of claim 19, wherein the generation unit is configured to calculate a normalization threshold using the parameter value and the at least some optionally selected error-based weight scores and to generate the weight label of each of the prediction models using the normalization threshold, the parameter value and the error-based weight scores for the prediction models.
